(19) United States
(12) Patent Application Publication
(10) Pub. No.: US 2024/0085083 A1
(43) Pub. Date: Mar. 14, 2024

(54) Touchless Compartmentalized Cooler

(71) Applicant: Coold LLC, Flower Mound, TX (US)

(72) Inventor: Randolph Charles Shaw, Frisco, TX (US)

(21) Appl. No.: 17/941,437

(22) Filed: Sep. 9, 2022

(51) Int. Cl. F25D 13/04; A61L 2/10; F25D 23/02

(52) U.S. Cl. CPC F25D 13/04; A61L 2/10; F25D 23/028; A61L 2202/122; F25D 2400/02

(57) ABSTRACT

A touchless compartmentalized cooler includes set of individual refrigeration cubbies with private/securable access for individual users. It allows a person to access a cubby to place food/beverages into a single cubby with no other people's items. It is lockable by the user to keep their items secure until they unlock the cubby to access their items or permanently remove them. The use of the touchless compartmentalized cooler can be entirely contactless by using the mobile app associated with the touchless compartmentalized cooler or can be accessed on the single control panel on, or near, the touchless compartmentalized cooler.

### FIELD OF THE DISCLOSURE

The present disclosure generally relates to a refrigerator. More particularly, the present disclosure relates to systems and methods for a touchless compartmentalized cooler.

### BACKGROUND OF THE DISCLOSURE

There is a need for offices and other workplaces to change due to the pandemic. For example, in existing breakrooms or the like, there is a shared refrigerator or cooler where people can place their lunch, drinks, etc. This is a shared device with everyone's items together and with each person touching the handle and accessing the same space. With social distancing and the like associated with the pandemic, there is a need to provide touchless and separation of space in a cooler/refrigerator.

### BRIEF SUMMARY OF THE DISCLOSURE

In various embodiments, the present disclosure includes a method having steps, a system including at least one processor and memory with instructions that, when executed, cause the at least one processor to implement the steps, and a non-transitory computer-readable medium having instructions stored thereon for programming at least one processor to perform the steps.

### BRIEF DESCRIPTION OF THE DRAWINGS

The present disclosure is illustrated and described herein with reference to the various drawings, in which like reference numbers are used to denote like system components/method steps, as appropriate, and in which:

FIG. 1 is a front view of a compartmentalized cooler.

FIG. 2 is a rear view of the compartmentalized cooler.

FIG. 3 is a side view of the compartmentalized cooler.

FIG. 4 is another side view of the compartmentalized cooler.

FIG. 5 is a top view of the compartmentalized cooler.

FIG. 6 is a front perspective view of the compartmentalized cooler.

FIG. 7 is another front perspective view of the compartmentalized cooler.

FIG. 8 is another front perspective view of the compartmentalized cooler.

FIG. 9 is a rear perspective view of the compartmentalized cooler.

FIG. 10 is another rear perspective view of the compartmentalized cooler.

FIG. 11 is another front perspective view of the compartmentalized cooler.

FIG. 12 is another front perspective view of the compartmentalized cooler.

FIG. 13 is another front perspective view of the compartmentalized cooler.

FIG. 14 is another front perspective view of the compartmentalized cooler.

FIG. 15 is another rear perspective view of the compartmentalized cooler.

### DETAILED DESCRIPTION OF THE DISCLOSURE

In various embodiments, the present disclosure relates to systems and methods for a touchless compartmentalized cooler with private/securable access for individual users. It allows a person to access a cubby to place food/beverages into a single cubby with no other people's items. It is lockable by the user to keep their items secure until they unlock the cubby to access their items or permanently remove them. The use of the touchless compartmentalized cooler can be entirely contactless by using a mobile app associated with the touchless compartmentalized cooler or can be accessed on the single control panel on, or near, the touchless compartmentalized cooler apparatus.

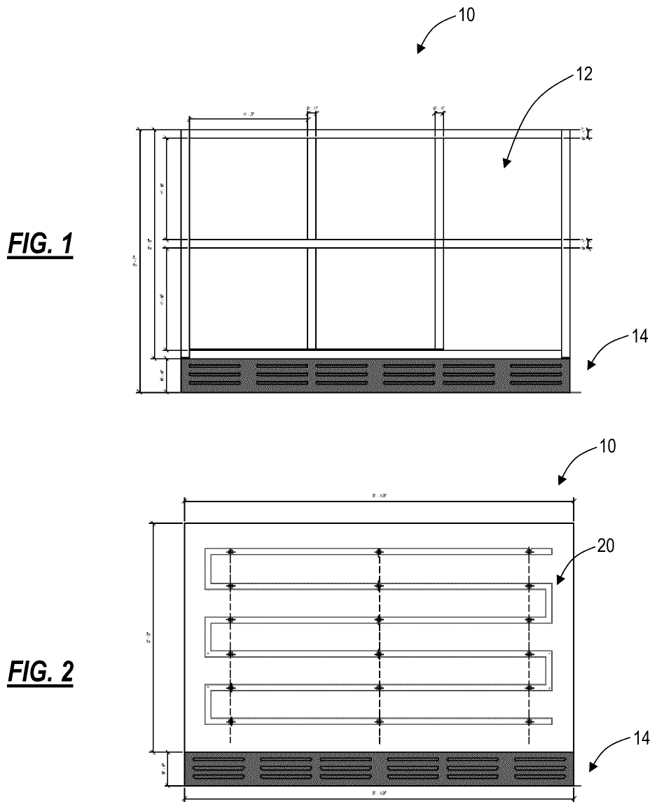

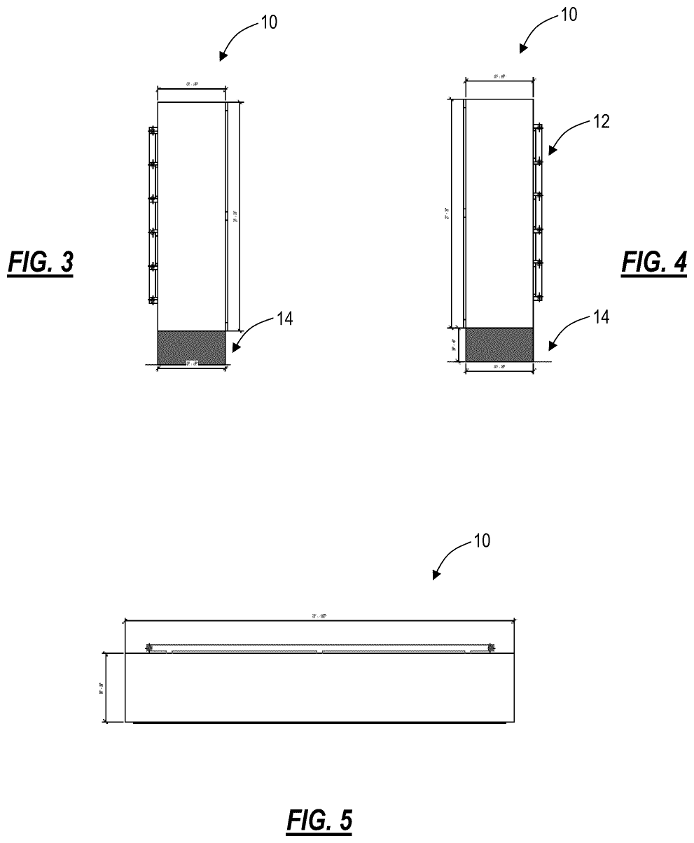

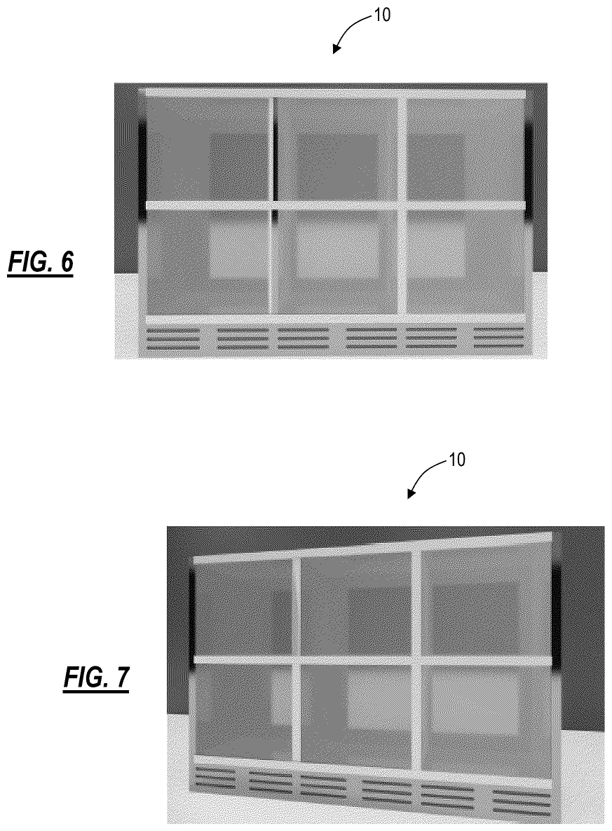

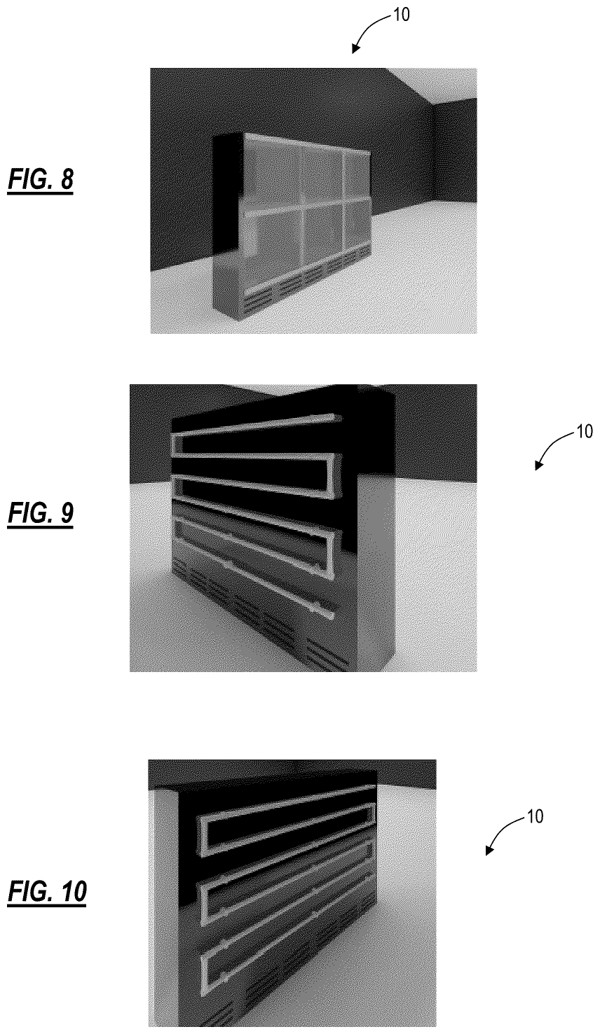

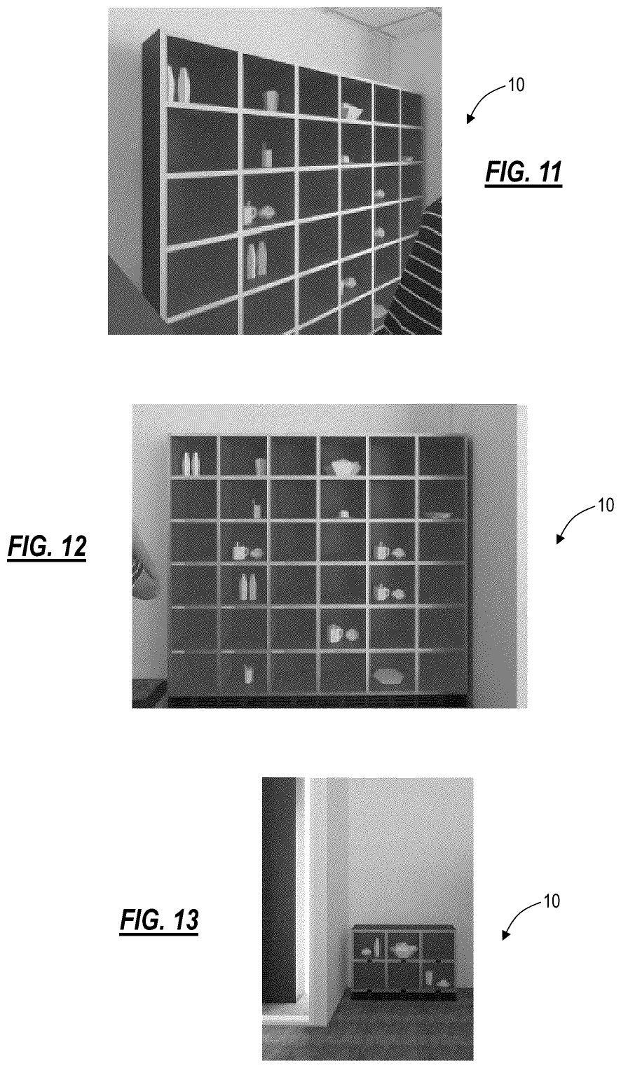

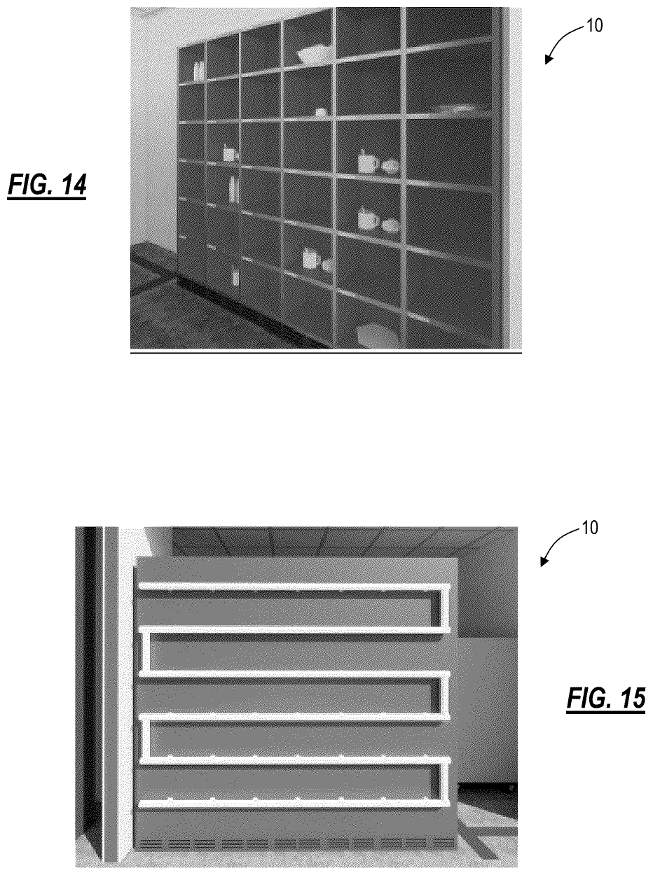

FIGS. 1-15 are various views of a touchless compartmentalized cooler 10 with a plurality of cubes 12. Each cube 12 can be optimally sized to accommodate typical user food/beverage containers without excess space to minimize size/dimension of entire system. For example, each cube 12 can be approximately 8 inches wide by 12 inches tall by 8 inches deep or similar size. Each cube 12 may be rectangular shaped, hexagonal, or other shapes as determined for maximum operational, storage, and aesthetic value.

The touchless compartmentalized cooler 10 can have various configurations such as, for example, 3×3 cubbies, 2×2, 3×2, 5×6, 5×30, etc. FIGS. 1-15 illustrate different configurations. As described herein, the cubes 12 can also be referred to as cubbies. Also, with the configurations, the touchless compartmentalized cooler 10 can be modular and different sizes can be achieved by connecting smaller units. The touchless compartmentalized cooler 10 may also be custom-configured for unique shapes as desired. The configurations may vary to factor in weight, dimension, etc for transport, freight elevator size limits, installation labor, safety, etc. Also, the touchless compartmentalized cooler 10 can be shipped in modules and installed on-site.

The touchless compartmentalized cooler 10 includes a base 14 designed to adequately support the weight and stabilize the structure for safety. May/may not be secured against the wall structure. The material will be adequate to secure the cubbies from entry, handle the weight, and contain the cooled air. The material may be painted/coated per user preference for aesthetic design/branding.

A centralized electrical/mechanical system 20 will be placed optimally behind the cubbies obscured and protected by a metal or similar material to minimize noise/exposure to users of equipment. They may be placed optimally for performance as well as ease for maintenance/service access. Also may be placed for optimal balance of weight for the units.

Each Cubby will be labeled with a Cubby number, form factor can be anything. Each Cubby will have an indicator light or mechanism to communicate to the user that the cubby is occupied/in-use or open/available. Inside surface may be standard materials or other as necessary for improved look, feel, cleaning, germ minimization, etc. A front door may be a variety of materials but adequate to contain the cooled temperatures. Examples include transparent glass, Plexiglas, frosted/opaque materials as well as paintable material allowing the surface to be an art-piece, mosaic, mural, etc.

Central Electrical System (back end) for all Cubbies is designed with adequately rated power to properly cool each cubby to FDA or other industry standard temperature and humidity ranges. The electrical system is properly grounded and rated for code compliance. The electrical system may contain standard LED lighting or Anti-microbial light or UVC light in each Cubby for sanitation against germs. There can be colored light configuration options in each Cubby for various uses (decorative, occupied/available, out of service). Red/Green (or other) light indicator for occupied/available

Electricity may be supplied to a motorized apparatus to open the front door of each cubby without needing to be touched by the user. May open in any way. roll/rotate/swing up, down, sideways, etc. The electrical system may contain intelligence to reduce energy/cooling load in a power-saver mode for un-used units. One (1) central controller will be digital for accessing/security coding cubbies in lieu of mobile app. May be installed on unit or separately and connected wired/wirelessly. Either with the app or controller, user will select cubby #, enter a personal code of their choice to open the cubby, insert items, then re-enter code to lock cubby. Or some similar process. Various other intelligence may be included in the app or controller for various functions i.e. user-controlled temperature ranges (within an approved preset limit for code, FDA, etc.) admin rights/override to open cubbies for disposal, cleaning, etc. communications with user phones of when to remove for needed cleaning, left in too long, etc.

The touchless compartmentalized cooler 10 can include a central mechanical System (back end) for all Cubbies. Designed with adequately rated power to properly cool each cubby to FDA or other industry standard temperature and humidity ranges. Properly grounded and rated for code compliance. May contain standard LED lighting or Anti-microbial light or UVC light in each Cubby for sanitation against germs. Colored light configuration options in each Cubby for various uses (decorative, occupied/available, out of service). Red/Green light indicator (or other) for occupied/available. One (1) central controller will be digital for accessing/security coding cubbies in lieu of mobile app. It would have the numbers for establishing codes and locking/unlocking cubby spaces similar to the smart phone app. May elect this option. It may be designed to attach directly to the cubby, or may attach to a nearby wall or surface and be connected wired/wirelessly to the cooler cubby. Electricity may be supplied to a motorized apparatus to open the front door of each cubby without needing to be touched by the user. May open in any way roll/rotate/swing up, down, sideways, etc.

The cubby may function as a cooler and/or a warmer using the exact same parts. The cubby may be a warmer or cooler at the entire unit level or possibly the individual cubby level. The mechanical/electrical systems may be unique and/or isolated to each individual cubby. The cubby may integrate sustainability features including reduced electrical draw, renewable energy, solar, etc. The outside materials on the cubby may be painted, or wrapped with graphics, logos, artwork or any kind for decorative and artistic purposes. May be made of any suitable material to meet warming/cooling functionality as well as other benefits i.e. lighter weight, durability, etc. The cubby doors may open by any means as desired or designed for effectiveness. The mobile may access cubbies using a QR code or similar as additional functionality. The Mobile App may be customizable to make unique for each client logo/imagery, etc.

### Mobile App

The touchless compartmentalized cooler 10 may have One (1) central controller will be digital for accessing/security coding cubbies in lieu of mobile app. May be installed on unit or separately and connected wired/wirelessly. Either app or controller, user will select cubby #, enter a personal code of their choice to open the cubby, insert items, then re-enter code to lock cubby. Or some similar process.

Various other intelligence may be included in the app or controller for various functions i.e. user-controlled temperature ranges (within an approved preset limit for code, FDA, etc.) admin rights/override to open cubbies for disposal, cleaning, etc. communications with user phones of when to remove for needed cleaning, left in too long, etc.

### Description and Functionality

| ITEM | DESCRIPTION |
|---|---|
| 1. App Icon | Cool logo/colors/image |
| 2. Home Page | Login (user name field and password field), |
| | forgot login, etc. |
| 3. Unit Mapping Page | Map to available Cooler Cubby UNITS (Unit #, |
| | permitted, location) |
| 4. Cubby Unit Page | Image of cubbies by unit number (grayed out if |
| | occupied) |
| | Instructions to select available cubby |
| | Tap or enter Cubby selected |
| | Once cubby selected, enter 4-digit code to unlock |
| | Cubby opens |
| | Put items in cubby and close |
| | Re-enter 4-digit code to lock |
| 5. Redo #1-4 above | Unlock Cubby be re-entering 4-digit code |
| | Take out items |
| 6. Cubby Unit Page | That cubby is grayed out and disabled from use |
| | until cleaned |

### Back End Funciontality

a. Administrator Functions
I. Set automated push notifications to remind users of food at set durations
II. Send individual users notifications
III. Send user wide notifications (remove food by x time/date, etc.)
IV. Send automatic notifications to admin/cleaning crew to clean cubby once #5 above is complete
V. Allow admin/cleaning crew to re-enable use of cubby once cleaned. Resets unit on #4 above
VI. Universal access code to unlock any/all cubbies at once for deep cleaning
VII. Track usage of cubbies
VIII. Track energy usage, performance metrics, maintenance needs (possible with sensors on HW)
IX. Map/track all cubby units across facility or portfolio of facilities
X. Others

It will be appreciated that some embodiments described herein may include or utilize one or more generic or specialized processors (“one or more processors”) such as microprocessors; Central Processing Units (CPUs); Digital Signal Processors (DSPs): customized processors such as Network Processors (NPs) or Network Processing Units (NPUs), Graphics Processing Units (GPUs), or the like; Field-Programmable Gate Arrays (FPGAs); and the like along with unique stored program instructions (including both software and firmware) for control thereof to implement, in conjunction with certain non-processor circuits, some, most, or all of the functions of the methods and/or systems described herein. Alternatively, some or all functions may be implemented by a state machine that has no stored program instructions, or in one or more Application-Specific Integrated Circuits (ASICs), in which each function or some combinations of certain of the functions are implemented as custom logic or circuitry. Of course, a combination of the aforementioned approaches may be used. For some of the embodiments described herein, a corresponding device in hardware and optionally with software, firmware, and a combination thereof can be referred to as “circuitry configured to,” “logic configured to,” etc. perform a set of operations, steps, methods, processes, algorithms, functions, techniques, etc. on digital and/or analog signals as described herein for the various embodiments.

Moreover, some embodiments may include a non-transitory computer-readable medium having instructions stored thereon for programming a computer, server, appliance, device, at least one processor, circuit/circuitry, etc. to perform functions as described and claimed herein. Examples of such non-transitory computer-readable medium include, but are not limited to, a hard disk, an optical storage device, a magnetic storage device, a Read-Only Memory (ROM), a Programmable ROM (PROM), an Erasable PROM (EPROM), an Electrically EPROM (EEPROM), Flash memory, and the like. When stored in the non-transitory computer-readable medium, software can include instructions executable by one or more processors (e.g., any type of programmable circuitry or logic) that, in response to such execution, cause the one or more processors to perform a set of operations, steps, methods, processes, algorithms, functions, techniques, etc. as described herein for the various embodiments.

Although the present disclosure has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present disclosure, are contemplated thereby, and are intended to be covered by the following claims. Moreover, it is noted that the various elements, operations, steps, methods, processes, algorithms, functions, techniques, etc. described herein can be used in any and all combinations with each other.

What is claimed is:

1. A touchless compartmentalized cooler comprising:
a base;
a housing supported by the base;
a plurality of cubbies in the housing, each cubby including a door on a front side of the housing;
a centralized electrical and mechanical system located on a rear side of the housing, wherein the centralized electrical and mechanical system is coupled to each of the plurality of cubbies and configured to any of warm and cool each cubby independently; and
a touchless mechanism to open the doors of each of the plurality of cubbies.

2. The touchless compartmentalized cooler of claim 1, wherein the housing is a first housing, and further comprising
a second housing having a second plurality of cubbies, wherein the second housing is connectable to the first housing.

3. The touchless compartmentalized cooler of claim 1, further comprising
anti-microbial light or Ultraviolet-C (UVC) light in each cubby for sanitation against germs.

4. The touchless compartmentalized cooler of claim 1, wherein the touchless mechanism includes a centralized controller in communication with the centralized electrical and mechanical system.

5. The touchless compartmentalized cooler of claim 1, wherein the touchless mechanism includes a mobile application on a user device in communication with the centralized electrical and mechanical system.

6. The touchless compartmentalized cooler of claim 5, wherein the mobile application controls the electrical and mechanical system.

7. The touchless compartmentalized cooler of claim 6, wherein the controls include temperature ranges for each of the plurality of cubbies.

8. The touchless compartmentalized cooler of claim 6, wherein the controls include accessing each of the plurality of cubbies.

9. The touchless compartmentalized cooler of claim 1, wherein at least two of the plurality of cubbies are at different temperature settings.

10. The touchless compartmentalized cooler of claim 1, wherein some of the plurality of cubbies are for cooling and others of the plurality of cubbies are for warming.
